(19) United States
(12) Patent Application Publication
(10) Pub. No.: US 2005/0020693 A1
(43) Pub. Date: Jan. 27, 2005

(54) Pharmaceutical preparations useful as drying agents and for treatment of warts

(76) Inventor: Richard Strauss, Woodbury, NY (US)

Correspondence Address: DARBY & DARBY P.C., P. O. BOX 5257, NEW YORK, NY, 10150-5257, US

(21) Appl. No.: 10/627,241

(22) Filed: Jul. 24, 2003

(51) Int. Cl. A61K 31/115

(52) U.S. Cl. 514/694

(57) ABSTRACT

Pharmaceutical preparations are provided for directly treating a skin growth, such as a wart, or as drying agents for pre and post surgical removal of the skin growth or for non-surgical laser treatment of the skin growth where dryness is required. In one exemplary embodiment, the composition includes formaldehyde as an active ingredient, wherein the formaldehyde is present in an amount from about 2% to about 20% by weight and it includes at least one non-active ingredient and wherein the composition is in the form of a gel or a powder.

### TECHNICAL FIELD

The present invention relates to pharmaceutical preparations and more particularly, to pharmaceutical preparations that safeguard against offensive odor and dry excessive moisture at an application site, such as the feet.

### BACKGROUND

Human skin and mucousal membranes support a wide range of growth abnormalities which exhibit a wide range of sizes, shapes and colors. Although many of the these growth abnormalities are not adverse to the health of the patient, they are in any event frequently cosmetically unappealing and as such are often times a cause of great discomfort. Moreover, these growth abnormalities are also prone to injury and infection and can be a source of a great deal of discomfort and pain due to the size and/or location of the growth abnormalities.

One common growth abnormality that most people are familiar with is warts. Warts are non-cancerous skin growth cause by a viral infection in the top layer of the skin or mucous membranes. Viruses that cause warts belong to a “family” called human papillomavirus (HPV). The appearance of a wart depends upon where the wart is growing. Warts are typically skin colored and feel rough to the touch, but warts can likewise be dark, flat or smooth. There are at least 60 types of HPV viruses and warts can grow on all parts of your body. The more common areas where warts may grow are the hands and feet of a patient. Various types of warts may form on one or more of these regions along with other regions of the body. For example, some types of warts which commonly grow on a foot are plantar warts, mosaic warts, and multiple warts.

There are a number of different treatments for dealing with the presence of one or more warts on the body. For example, one common treatment is the repeated application of salicylic acid on the warts and then subsequent filing away of the dead skin of the warts. Another treatment is the application of cantharidin to the warts. The doctor will “paint” this chemical onto the wart which is then covered with a bandage for a time period before the doctor removed the dead skin of the wart. Yet another treatment is the application of liquid nitrogen to freeze the wart in a procedure commonly known as cryotherapy. Generally, a number of cryotherapy visits is required before improvement is noted and the wart is removed. It will be appreciated that there are a number of other treatments for removing a wart from the body. For example, warts on the skin can be removed by burning the wart, cutting out the wart and removing the wart with a laser.

While these treatments have varying levels of success, there are a number of disadvantages associated with each of them. For example, a number of these treatments require multiple visits and some require a doctor to perform the operation as opposed to a patient applying a medicament to the wart itself. In addition, a number of the over counter products and even some products that doctors dispense are difficult to use since they are liquid in form and therefore, they tend to run when the patient places a drop on the wart. This can lead to an ineffective treatment and product waste since the liquid medicament can easily run off the desired application site, i.e., the wart itself, and onto healthy skin which can become damaged. It is desired for the medicament to remain on top of and cover the wart both during and post treatment to ensure that the medicament properly works. While there are some patch like structures available for delivering a medicament, such as salicylic acid, the effectiveness of these products is questionable and also, the patch may lift or otherwise become removed from the skin of the patient, thereby reducing the effectiveness of the treatment.

What has heretofore not been available is an improved compound and method for treating abnormal growths, particularly warts, that is not only easy to apply but also remains localized at the site of the abnormal growth.

### SUMMARY

Pharmaceutical preparations are provided for directly treating a skin growth, such as a wart, or as drying agents for pre and post surgical removal of the skin growth or for non-surgical laser treatment of the skin growth where dryness is required. In one exemplary embodiment, the composition includes formaldehyde as an active ingredient, wherein the formaldehyde is present in an amount from about 2% to about 20% by weight and it includes at least one non-active ingredient and wherein the composition is in the form of a gel or a powder.

The preparations can include one or more of the following: thickening agents, surfactants/spreading agents, alcohols, fragrances and other support ingredients, such as clay, etc. Some exemplary materials that have the aforementioned characteristics are carbomer, polysorbate 20, isopropanol, triethanolamine, peppermint oil, corn starch, and kaolin.

The present pharmaceutical preparations provide an attractive alternative to conventional preparations that are intended to treat warts. The quick drying nature of the preparation which is preferably in either a gel or powder form advantageously permits the preparation to be easily applied to a skin growth, such as a wart. Since the preparation is not in liquid form, it can be easily stored and applied to the skin growth.

Further aspects and features of the exemplary actuator and method of manufacture thereof can be appreciated from the appended Figures and accompanying written description.

### DETAILED DESCRIPTION OF PREFERRED EMBODIMENTS

The present pharmaceutical preparations are useful for treating an abnormal skin grown, such as a wart. In particular, the present pharmaceutical preparations are useful for treating warts that are present on the body of a patient, such as in the hand and foot locations.

The pharmaceutical preparation includes as an active ingredient formaldehyde, such as a formaldehyde 37% solution, which acts as a drying agent and is useful in treating warts when the preparation is applied thereon. It will be appreciated that formaldehyde 37% is merely one product concentration and formaldehyde solution comes in any number of other concentrations, e.g., it comes in a more concentrated version and also can be diluted. In one embodiment, the pharmaceutical preparation is the active medicine that is applied to the wart for treatment, reduction, and removal of the wart. In other words, according to this one embodiment, the pharmaceutical preparation is applied directly to the wart without any other primary surgical procedure or treatment being done for removing the wart. As the active ingredient, formaldehyde serves to dry the wart tissue and then the treating physician subsequently files away the dead skin of the wart.

In this embodiment, where the preparation is used as the principal agent to treat and remove the wart, the preparation is preferably provided in a form other than a liquid and more specifically, the present pharmaceutical preparation is provided in paste (ointment or cream) or gel form by mixing the active ingredient(s) of the pharmaceutical preparation with suitable thickening agents or gelating materials or other materials which support the active ingredient, the likes of which are well know in the art. Furthermore, the pharmaceutical preparation can alternatively be provided in a rehydratable powder form, such as a tincture.

In the gel and powder form, the active formaldehyde is preferably present in a weight/volume ratio of between about 2% to about 20% and preferably from about 5% to about 15%, e.g., about 10%. The remaining amount of the pharmaceutical preparation is formed of non-active ingredients, such as thickening agents and the like and ingredients to add fragrance and/or color to the preparation. For example, in the paste or gel form, deionized water is present in an amount of about 50% or greater by weight/volume ratio and in the powder form, silica is present in an amount of 45% or greater by weight/volume ratio. The particle size of the silica is variable; however, it should be of a size that supports the formaldehyde solution and permits the composition to assume a powder like form. In one exemplary embodiment, the silica has a particle size of about 0.01 micron.

In either of these embodiments, the gel or powder form of the preparation permits it to be applied directly to the wart and unlike conventional liquid wart removal solutions, the viscous nature of the present gel and the solid nature of the powder increase the likelihood and ensure that the preparation remains on the application site (the wart) after it has been applied. This is contrast to a number of conventional products which, as described above, have a tendency to run off the wart onto healthy tissue.

Formaldehyde is a particularly effective drying agent that is an excellent topical agent for drying out skin and particularly drying out an undesirable skin growth, such as a wart. By quickly and effectively drying out the wart, the wart is treated and then can subsequently be reduced in size by filing the dry dead skin. This process is repeated as often as necessary to reduce the wart more and more until the wart is finally removed.

As previously mentioned, the pharmaceutical preparations include a number of other non-active ingredients that give the preparations their desired form, e.g., gel, powder, etc. For example, the preparations can include one or more of the following: thickening agents, surfactants/spreading agents, alcohols, fragrances and other ingredients. Some exemplary materials that have the aforementioned characteristics are carbomer, polysorbate 20, isopropanol, triethanolamine, peppermint oil, corn starch, and kaolin (a clay). It will be appreciated that these materials are merely in nature and other suitable or equivalent materials can be substituted and used. For example, carbomer is one suitable thickener; however, there are a number of other type of suitable thickeners, such as methylcellulose, CMC, tragacanth gum, xanthan gum, etc. Similarly, other alcohols can be substituted for isopropyl alcohol.

In another embodiment, the present pharmaceutical preparations can be used as a drying agent for pre and post surgical removal of warts, or for non-surgical laser treatment of warts where dryness is required. The preparations safeguard against offensive odors and dry excessive moisture of the feet. Thus, in this embodiment, the present pharmaceutical preparation is used either prior to and/or after another treatment (surgical) is performed to remove the wart. In most if not all of the surgical type treatments, such as cutting, burning or using a laser, it is desirable for the surgical site to be free of excessive moisture. Since the present preparation acts as a drying agent, it is particularly suited for application to the surgical site either pre or post surgery. In this embodiment, the preparation is applied in a similar manner as in the previous embodiment, in that the preparation is placed on the tissue at the surgical site. Because the preparation has a fragrance ingredient, it safeguards against offensive odors as well as drying any excessive moisture at the surgical site due to the formaldehyde.

Advantageously, the drying agent characteristics of the present preparations permit it to dry fast in either the gel form or the powder to form which results in a residue being left on the wart itself to promote the treatment thereof. The preparations are formulated so that in any of their forms, the preparations dry fast on the skin and dry the skin to which they are applied.

The examples which follow, in combination with the above descriptions, are intended to illustrate the present pharmaceutical preparation in more detail and in a non limiting fashion.

### EXAMPLE 1

In this example, the pharmaceutical preparation is in the form of a gel that is intended to be placed on the abnormal growth, e.g., a wart. In this example, a 500 lbs batch size is produced; however, it will be understood that the batch size is variable depending upon the individual needs of the manufacturer. Initially, 295 lbs of deionized water is placed into a mixing vessel. 5.0 lbs of carbomer (e.g., Carbopol Ultrez commercially available from Noveon, Inc.) is sprinkled on top of the deionized water and the mixture is left standing until the carbomer is saturated. In a separate vessel, 7.5 lbs of peppermint oil is mixed with 37.5 lbs of polysorbate 20 (e.g., Tween 20), which is a surfactant and spreading agent. This solution is set aside. Once the carbomer is saturated, the mixture in this vessel is mixed (stirred) and then 2.5 lbs of triethanolamine is added. The mixture will begin to thicken as it is mixed. The mixing is continued and the polysorbate 20/peppermint oil solution and the mixing is continued. Next, 142.5 lbs of formaldehyde 37% and 10.0 lbs of isopropanol are added to the mixture and the mixing continues until it is uniform and the final gel product is thus formed. The gel product has a final pH of about 4.75.

Table 1 below lists the above ingredients in terms of their weight/volume percentages to permit a user to make any size batch since these percentages need only be followed when manufacturing a batch.

TABLE 1
Formula for thickened for formaldehyde gel
(batch size 500.00 lbs)

| Ingredient | Weight/volume % | Quantity (lbs) |
|---|---|---|
| Deionized water | 59.00 | 295.0 |
| Formaldehyde 37% | 28.5 | 142.5 |
| Polysorbate 20 | 7.50 | 37.50 |
| Isopropanol | 2.0 | 10.0 |
| Carbomer | 1.00 | 5.0 |
| Peppermint oil | 1.50 | 7.5 |
| Triethanolamine | 0.50 | 2.5 |

When the pharmaceutical preparation is in the form of a gel, the active ingredient is the formaldehyde 37%, while the rest of the above ingredients are inactive ingredients. The amount of active formaldehyde in the above preparation is about 10% by weight; however, it will be appreciated that it can be present in an amount from about 2% to about 20%.

### EXAMPLE 2

In this example, the pharmaceutical preparation is in the form of a powder that is intended to be placed on the abnormal growth, e.g., a wart. In this example, a 500 lbs batch size is produced; however, it will be understood that the batch size is variable depending upon the individual needs of the manufacturer. Because the preparation is in a solid form (powder), one of the major components is silica. One exemplary type of silica is commercially available from Charles B. Chrystal Co., Inc., New York, N.Y., under the name #32 precipitated silica, which is a material that is similar to smoked or fumed silica and is particularly useful as a material for thickening, flatting, flow control and absorption. In making a 10.00 lbs batch, 5.0 lbs of silica is placed into a container or the like. Next, 2.8 lbs of formaldehyde 37% and 1.0 lbs of peppermint oil are stirred into the silica. This mixture is stirred until it is uniform. Next, 0.8 lbs of corn starch and 0.4 lbs of kaolin are added and then the mixture is packaged into the appropriate packaging material or the like.

Table 2 below lists the above ingredients in terms of their weight/volume percentages to permit a user to make any size batch since these percentages need only be followed when manufacturing a batch.

TABLE 2
Formula for formaldehyde foot powder
(batch size 10.00 lbs)

| Ingredient | Weight/volume % | Quantity (lbs) |
|---|---|---|
| Silica | 50.0 | 5.0 |
| Formaldehyde 37% | 28.0 | 2.8 |
| Peppermint oil | 10.0 | 1.0 |
| Corn starch | 8.0 | 0.8 |
| Kaolin | 4.0 | 0.4 |

The amount of active formaldehyde in the above preparation is about 10% by weight; however, it will be appreciated that it can be present in an amount from about 2% to about 20%.

For both examples 1 and 2, it will be understood that the concentrations of the different ingredients can be varied in order to create a final product that has certain desired characteristics. For example, the concentration of thickeners can be varied depending upon the desired viscosity.

The present pharmaceutical preparations provide an attractive alternative to conventional preparations that are intended to treat warts. The quick drying nature of the preparation which is preferably in either a gel or powder form advantageously permits the preparation to be easily applied to a skin growth, such as a wart. Since the preparation is not in liquid form, it can be easily stored and applied to the skin growth.

It will be appreciated by persons skilled in the art that the present invention is not limited to the embodiments described thus far with reference to the accompanying drawings; rather the present invention is limited only by the following claims.

1. A composition for treating a wart comprising:
formaldehyde as an active ingredient, wherein the formaldehyde is present in an amount from about 2% to about 20% by weight;
at least one non-active ingredient; and
wherein the composition is in the form of a gel.

2. The composition of claim 1, wherein the formaldehyde is present in an amount greater than or equal to about 10% by weight.

3. The composition of claim 1, wherein the formaldehyde is present in an amount between about 5% to about 15% by weight.

4. The composition of claim 1, wherein the at least one non-active ingredient includes water which is present in amount of at least 50% by weight.

5. The composition of claim 4, further including thickening agent in an amount sufficient to cause the composition to be in gel form.

6. The composition of claim 1, wherein the at least one non-active ingredient includes deionized water, polysorbate 20, isopropanol, carbomer, peppermint oil and triethanolamine.

7. The composition of claim 6, wherein the polysorbate 20 is present in an amount greater than 5% by weight.

8. The composition of claim 6, wherein each of the isopropanol, carbomer, peppermint oil and triethanolamine is present in an amount less than 5% by weight.

9. The composition of claim 1, wherein the at least one non-active ingredient includes at least one thickening agent, a fragrance and an alcohol.

10. A composition for treating a wart comprising:
formaldehyde as an active ingredient, wherein the formaldehyde is present in an amount greater than or equal to about 10% by weight;
non-active ingredients including at least two ingredients selected from the group consisting of deionized water, polysorbate 20, isopropanol, carbomer, peppermint oil and triethanolamine; and
wherein the composition is in the form of a gel.

11. The composition of claim 10, wherein the deionized water is present in an amount greater than about 58%.

12. The composition of claim 10, wherein the polysorbate 20 is present in an amount greater than 5% by weight.

13. The composition of claim 10, wherein each of the isopropanol, carbomer, peppermint oil and triethanolamine is present in an amount less than 5% by weight.

14. A composition for treating a wart comprising:
formaldehyde as an active ingredient, wherein the formaldehyde is present in an amount from about 2% to about 30% by weight;
at least one non-active ingredient; and
wherein the composition is in the form of a powder.

15. The composition of claim 14, wherein the formaldehyde is present in an amount greater than or equal to about 10% by weight.

16. The composition of claim 14, wherein the at least one non-active ingredient includes a thickening agent present in an amount greater than or equal to about 50%.

17. The composition of claim 16, wherein the thickener comprises silica.

18. The composition of claim 14, wherein the at least one non-active ingredient includes peppermint oil, corn starch and kaolin.

19. A method of treating a wart comprising the steps of:
providing a pharmaceutical preparation that includes formaldehyde as an active ingredient and at least one non-active, wherein the formaldehyde is present in an amount from about 2% to about 20% by weight and the composition is in the form of a powder or a gel;
applying the preparation to a surface of the wart to cause drying of the wart; and
subsequently removing the dried wart tissue.

20. The method of claim 19, wherein the formaldehyde is present in amount greater than or equal to about 10% by weight.

21. The method of claim 19, wherein the at least one non-active ingredient includes a thickening agent present in an amount greater than or equal to about 50%.

22. The method of claim 21, wherein the thickening agent comprises silica.

23. The method of claim 21, wherein the non-active ingredients permit the composition to be in the form of a gel, wherein the non-active ingredients include at least three ingredients selected from the group consisting of deionized water; polysorbate 20, isopropanol, carbomer, peppermint oil and triethanolamine.

24. A method of treating a surgical site prior to or after another procedure is performed to remove a skin growth, the method comprising the steps of:
providing a pharmaceutical preparation that includes formaldehyde as an active ingredient and at least one non-active ingredient, wherein the formaldehyde is present in an amount from about 2% to about 20% by weight and the composition is in the form of a powder or a gel;
applying the preparation to the surgical site to remove excessive moisture at the surgical site; and
performing the other procedure to treat the skin growth in the case where the preparation is used as a pretreatment prior to the other procedure being performed.

25. The method of claim 24, wherein the other procedure comprises a pre or post surgical procedure for removal of the skin growth or non-surgical laser treatment of the skin growth.

26. The method of claim 24, wherein the skin growth comprises one or more warts.

27. The method of claim 24, wherein the formaldehyde is present in amount between about 5% to about 10% by weight.
